(19) United States
(12) Patent Application Publication
(10) Pub. No.: US 2025/0011636 A1
(43) Pub. Date: Jan. 9, 2025

(54) HYBRID METAMATERIALS

(71) Applicant: CASE WESTERN RESERVE UNIVERSITY, Cleveland, OH (US)

(72) Inventors: Yusuf Dikici, Cleveland Heights, OH (US); Ozan Akkus, Shaker Heights, OH (US)

(21) Appl. No.: 18/613,999

(22) Filed: Mar. 22, 2024

Related U.S. Application Data
(60) Provisional application No. 63/491,618, filed on Mar. 22, 2023.

(51) Int. Cl. C09K 3/00

(52) U.S. Cl. CPC C09K 3/00

(57) ABSTRACT

A hybrid metamaterial structure includes at least one auxetic portion and at least one non-auxetic portion connected to the auxetic portion. The at least one non-auxetic portion has a nodal honeycomb unit geometry.

### RELATED APPLICATIONS

This application claims the benefit of U.S. Provisional Application Ser. No. 63/491,618, field Mar. 22, 2023, the entirety of which is incorporated by reference herein.

### GOVERNMENT FUNDING

This invention was made with government support under 1652839, 2047330, and 1743475 awarded by the National Science Foundation. The government has certain rights in the invention.

### TECHNICAL FIELD

The present invention relates to metamaterials and, in particular, relates to hybrid metamaterial structures having auxetic and non-auxetic portions.

### BACKGROUND

The demand for complex and advanced material property needs in various applications has necessitated the development of novel functional materials. Recent advances in rapid prototyping and additive manufacturing have brought solutions for fabrication of architectured materials, thereby the number of studies on mechanical metamaterials have shown a significant increase. Metamaterials have been described as engineered materials with such properties that are rare or non-existent in the nature, and they are generally formed as patterns which consist of repeated cellular geometries.

These cells may be folding patterns as in origami, perforation patterns as in kirigami, or structure geometry as in lattices. Mechanical metamaterials are engineered materials of which mechanical properties primarily originate from their unit geometries and their mechanical properties can be customized without modifying chemical properties. Previous studies have investigated this concept for negative Poisson's ratio, negative compressibility, negative stiffness, negative swelling, elastic hysteresis, snapping deformations, out-of-plane deformation through 3D design of architectured metamaterials, and programmability.

Programmability is a unique feature of mechanical metamaterials, which is realized by adjusting local mechanical properties via tuning geometrical parameters. Previous studies used programmability of mechanical metamaterials to create a stiffness gradient, to control energy absorption characteristics, and also to obtain shape-morphing characteristics by adjusting geometrical parameters in order.

Auxetic metamaterials can be used in many engineering fields, including bioengineering. Some examples in the field of biomechanics are hip implant stem design, and bone screws.

A few studies on auxetics present hybrid structures which are created in order to combine mechanical advantages of auxetic and non-auxetic structures in the same pattern, such as obtainment of materials with zero Poisson's ratio and those studies mostly focuses on re-entrant-honeycomb combinations. However, hybridization of different material structures as such has been limited, mainly because stacking of these nodal structures is not straightforward.

### SUMMARY

With the above in mind, shape-morphing capabilities of metamaterials can be expanded by developing approaches that enable the integration of different types of cellular structures. To this end, a rational material design process is shown and described that fits together auxetic (e.g., anti-tetrachiral) and non-auxetic (e.g., nodal honeycomb) lattice structures with a shared grid of nodes to form a hybrid metamaterial structure having desired values of Poisson's ratios and Young's moduli. Through this scheme, deformation properties can be easily set piece-by-piece and 3D-printed in useful combinations.

For example, nodally-integrated tubular lattice structures can be formed to undergo worm-like peristalsis or snake-like undulations that result in faster insertion/deployment speeds than monophasic counterparts in narrow channels and in wider channels, respectively. In a certain scenario, such worm-like hybrid metamaterial structures can traverse between confined spaces that are otherwise impassable for the isotropic variant. These deformation mechanisms allow shape-morphing structures to be designed into customizable soft robot skins that have improved performance in confined spaces. The analytical material design approach described herein can make metamaterials more accessible for applications not only in soft robotics but also in medical devices or consumer products. Additionally, hybridizing auxetic and non-auxetic patterns through a computationally-driven design process may provide multiple shape-morphing characteristics, which may enable design of stents to match complex curviplanar morphologies, such as the gastrointestinal tract.

In accordance with an aspect of the invention, a hybrid metamaterial structure includes at least one auxetic portion and at least one non-auxetic portion connected to the auxetic portion. The at least one non-auxetic portion has a nodal honeycomb unit geometry.

In another aspect of the invention, the nodes are arranged in rows and columns.

In another aspect of the invention, the at least one auxetic portion has an anti-tetrachiral unit geometry.

In another aspect of the invention, the nodal honeycomb unit geometry includes individual honeycomb cells connected by rectangular nodes.

In another aspect of the invention, the nodal honeycomb unit geometry includes individual honeycomb cells connected by square nodes.

In another aspect of the invention, the nodal honeycomb unit geometry includes individual re-entrant honeycomb cells connected by rectangular nodes.

In another aspect of the invention, the at least one auxetic portion and the at least one non-auxetic portion cooperate to form a planar member.

In another aspect of the invention, the at least one auxetic portion and the at least one non-auxetic portion cooperate to form a tubular member.

In another aspect of the invention, the auxetic and non-auxetic portions are asymmetrically arranged about the structure.

In another aspect of the invention, the auxetic and non-auxetic portions have equal Young's Modulus values and equal but opposite Poisson's Ratio values.

In another aspect of the invention, the auxetic and non-auxetic portions are arranged in a checkerboard pattern to form a flexural member

In another aspect of the invention, the auxetic and non-auxetic portions are arranged in an alternating pattern along the length of the structure to form an expansional member.

In another aspect of the invention, the nodes interconnecting the unit cells of the non-auxetic portion are equally spaced along the same axis.

In another aspect of the invention, the structure comprises a planar sheet rolled into a tube and interconnected along longitudinal edges at the nodes.

In another aspect of the invention, a hybrid metamaterial structure includes at least one auxetic portion having an anti-tetrachiral unit geometry. At least one non-auxetic portion is connected to the auxetic portion and has a re-entrant honeycomb unit geometry interconnected by square nodes. The auxetic and non-auxetic portions have equal Young's Modulus values and equal but opposite Poisson's Ratio values.

In another aspect, a part for a soft robot or a medical device including a stent can be formed from the hybrid metamaterial structure.

Other objects and advantages and a fuller understanding of the invention will be had from the following detailed description and the accompanying drawings.

### BRIEF DESCRIPTION OF THE DRAWINGS

FIG. 1 is a schematic illustration of an example combined auxetic and non-auxetic structure.

FIG. 2A graphically illustrates the effects of geometrically and mechanically integrated unit cell geometry on Poisson's ratio.

FIG. 2B graphically illustrates the effects of geometrically and mechanically integrated unit cell geometry on Young's modulus.

FIG. 2C illustrates the different geometric parameter combinations used to generate FIGS. 2A-2B.

FIG. 2D is schematic illustrations of the template models having the geometric parameters of FIG. 2C.

FIG. 3A illustrates geometrical parameters of an anti-tetrachiral pattern with circular nodes.

FIG. 3B illustrates geometrical parameters of the anti-tetrachiral pattern of FIG. 3A with the circular nodes converted to square nodes.

FIG. 4A is a schematic illustration of geometrical parameters affecting mechanical properties of conventional honeycomb pattern.

FIG. 4B is a schematic illustration of a nodal honeycomb pattern created by expanding the thicknesses of the vertical ligaments in FIG. 4A to form square shape nodes.

FIG. 4C is a schematic illustration of the ligament intersection geometry of the pattern in FIG. 4B.

FIG. 5A is a table of various lattice geometries for tubular metastructures.

FIG. 5B is a schematic illustration of the interphase cell geometry for the tubular metastructure of FIG. 5A.

FIG. 5C is a schematic illustration of a tubular metastructure having an expansional configuration.

FIG. 5D is a schematic illustration of a tubular metastructure having a flexural configuration.

FIG. 5E graphically illustrates variations in the Poisson's ratio along the length of the metastructure in FIG. 5C.

FIG. 5F graphically illustrates variations in the bending behavior of the metastructure in FIG. 5D.

FIG. 6A is a schematic illustration of an example planar metastructure.

FIG. 6B is a schematic illustration of the metastructure of FIG. 6A formed into a tube.

FIG. 6C is a schematic illustration of the metastructure of FIG. 6B configured for implementation into a test machine.

FIG. 7A is a table of various lattice geometries for various tubular metastructures.

FIG. 7B is a schematic illustration of the tubular metastructures of FIG. 7A in both original and deformed states.

FIG. 7C graphically illustrates variations in the Poisson's ratio along the length of the metastructures in FIG. 7B.

FIG. 7D graphically illustrates variations in the bending behavior of the metastructures in FIG. 7B.

FIG. 8A is a schematic illustration of tubular metastructures within channels having a first configuration.

FIG. 8B graphically illustrates comparing movement of the metastructures within the channels having the first configuration.

FIG. 8C is a schematic illustration of tubular metastructures within channels having a second configuration.

FIG. 8D graphically illustrates comparing movement of the metastructures within the channels having the second configuration.

FIG. 8E is a schematic illustration of tubular metastructures within channels having a third configuration.

FIG. 8F graphically illustrates comparing movement of the metastructures of FIG. 8A within the channels having the third configuration.

FIG. 9A is a schematic illustrations of one example stacked metamaterial structure.

FIG. 9B graphically illustrates comparing the normalized Young's Modulus of different portions of the stacked metamaterial structure of FIG. 9A.

FIG. 10 illustrates the intersection of metacombination geometry and geometrical parameters.

FIG. 11 illustrates variation of Poisson's ratio with angle θ and corresponding h/l.

FIG. 12 illustrates Young's modulus distributions with θ and corresponding h/l for different ligament thicknesses in ATC.

FIG. 13A-13F illustrate a process for designing a deployed stent.

FIG. 14 is a schematic illustration depicting a process for defining deformation in a mesh-element tubular body.

FIG. 15 illustrates the comparison between the pre-defined canal and the deformed shape of the stent designed using the process of FIGS. 13A-13F.

### DETAILED DESCRIPTION

The present invention relates to metamaterials and, in particular, relates to hybrid, latticed metamaterial structures having cellular auxetic and non-auxetic portions. Lattice metamaterials enable designers to create materials with desired mechanical properties. These mechanical properties originate from the deformation mechanisms of their cellular structures. These mechanisms may provide Poisson's ratios beyond the theoretical limit of continuum materials. The most well-known lattice metamaterial is a honeycomb pattern, which has a positive Poisson's ratio. The analytical mechanical model of honeycomb has been studied and the deformation characteristics of its unit structure have been described as the combination of flexure, stretching, and hinging.

Combining auxetic and the non-auxetic lattices rationally may expand the design possibilities of shape-morphing structures and enable new applications that take advantage of local variations in material properties. However, while previous studies have shown that auxetic and non-auxetic lattices can be combined thanks to geometrical similarity and hybridization compatibility, e.g., for shape-matching to achieve zero Poisson's ratio, those studies were limited to only re-entrant and conventional honeycomb patterns, and wider applications, such as soft robotics, may require the advantages and benefits of other types cellular structures with a variety of deformation mechanisms. For instance, chiral models may offer options of isotropic, shear or twist deformation modes and the nodal component of the unit structures may provide easiness for practical applications.

To this end, the present invention provides an easy-to-implement, computationally inexpensive, analytical approach to systematically combine auxetic and non-auxetic lattices (AnA) that enable parametric design and modular construction of metastructures with nodal patterns. The developed analytical scheme can be implemented for creating AnA tubes of which auxetic and non-auxetic phases are programmed to generate the same magnitude of expansion and contraction under loading. Shape-morphing characteristics of a variety of combinations have been studied via finite element analysis (FEA) and mechanical testing, and the differences in design approaches for computational and experimental models investigated.

A proof-of-concept application has been demonstrated as crawling soft robot skins by using 3D-printed AnA tubes. The robots with AnA skins showed worm-like peristalsis or snake-like undulation motions under simple uniaxial actuation, depending on the combination configuration of auxetic and non-auxetic phases. Besides robotics, biomedical applications such as stents, or manufacturing applications, such as grippers, can be derived from this approach and are presented herein.

### Example 1

### Design Procedure

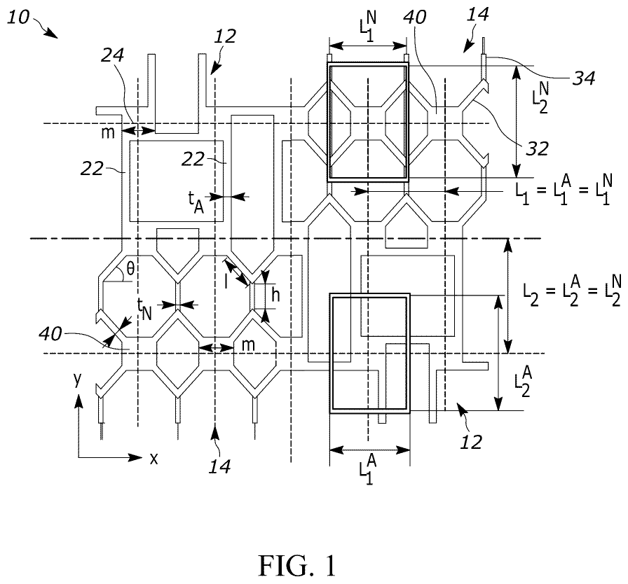

We aimed to connect auxetic and non-auxetic lattices over a space continuum. Spatial connectivity between two different lattice structures necessitates the geometrical spacings of the unit cells of the two lattices to be compatible. Furthermore, connectivity also necessitates that the unit cells have mutual geometrical features via which unit cells are grafted together. In this study, we chose a metamaterial structure 10 having an anti-tetrachiral pattern with square-shaped nodes as the auxetic phase/portion 12 and used their nodes as connecting elements with the non-auxetic phase/portion 14 constructed of nodal honeycomb (see FIG. 1).

In previous studies, nodes were defined as the circular, polygonal or elliptical features of the chiral cellular geometries. While anti-tetrachiral cellular structure has this nodal, geometrical component, none of the non-auxetic patterns that are present to date has nodes. Consequently, a “nodal honeycomb” pattern was developed and consisted of a modified version of conventional honeycomb. Through creation of nodal honeycomb, it became possible to execute a rational design procedure for combinations of anti-tetrachiral and nodal honeycomb lattices. Mathematical models explaining the deformation of these cellular structures are discussed at the end of this Example.

That said, the hybrid metamaterial structure 10 has a nodal honeycomb pattern 14 compatible for in-plane combinations with the anti-tetrachiral pattern 12 because both have nodes 24, 40 (at intersections of dashed lines) which is a mutual geometric feature in both cell structures. The unit cell boundaries are centered at the nodes 24, 40 and highlighted with black, rectangular frames. Each rectangular grid piece defined by dashed lines can be modularly replaced with either the anti-tetrachiral or the nodal honeycomb pattern 12, 14.

The anti-tetrachiral pattern 12 includes an array of ligaments or ligaments 22 interconnected at common nodes 24. Similarly, the honeycomb pattern 14 includes an array of angled ligaments 32 and straight ligaments 34. The angled ligaments 32 are interconnected to each other at common nodes 40.

Hybridization of two different patterns required design principles for geometric and mechanical integration. The first design principle was that the auxetic and non-auxetic portions 12, 14 should be spatially matched such that the nodes 24, 40 overlap. In other words, the cellular size of both portions 12, 14 and, thus, the distance between nodes 24, 40 should be equal.

$${L}_{1}^{A} = {L}_{1}^{N} \qquad ( 1 )$$

$${L}_{2}^{A} = {L}_{2}^{N} \qquad ( 2 )$$

A user-defined constraint defines the second principle in which the Poisson's ratio values of both lattice structures are set to be equal in magnitude. This constraint provides a symmetric basis for deformations of auxetic and non-auxetic portions 12, 14. In different iterations, this constraint can also be defined as different Poisson's ratio values for the two lattices to obtain asymmetry. For the sake of demonstration, we limited the analysis to symmetric Poisson's ratio values for the two portions 12, 14:

$${v}_{21}^{A} = - {v}_{21}^{N} \qquad ( 3 )$$

The third principle is that the Young's moduli for both portions 12, 14 are equal in order to distribute the applied displacement in each section equally, and to generate the same amplitudes of expansion or contraction for the auxetic and non-auxetic portions.

$${E}_{2}^{A} = {E}_{2}^{N} \qquad ( 4 )$$

The mathematical procedure based on these principles first takes predefined geometrical parameters (m, l, h, t_{N} in FIG. 1) to calculate the angle θ of the inclined ligaments 32 by using Equation 3. Poisson's ratios of unit cells are proportional to ratios of unit cells' lengths to their widths (see Equation 20 and Equation 27). Equation 3 can therefore be expressed as:

$$\frac{{L}_{2}^{A}}{{L}_{1}^{A}} = \frac{{L}_{2}^{N}}{{L}_{1}^{N}} \frac{- cos θ sin θ ( - \frac{1}{{K}_{f}} + \frac{1}{{K}_{s}} - \frac{1}{{K}_{h}} )}{\frac{{cos}^{2} θ}{{K}_{f}} + \frac{{sin}^{2} θ + ( h / l )}{{K}_{s}} + \frac{{cos}^{2} θ}{{K}_{h}}} \qquad ( 5 )$$

where K_{f}, Kₛ and Kₕ represent the stiffness values associated with flexure, stretching, and hinging of the ligaments connecting the nodes, respectively. Because of spatial-matching criteria (Equations 1 and 2), Equation 5 is simplified to:

$$\frac{- cos θ sin θ ( - \frac{1}{{K}_{f}} + \frac{1}{{K}_{s}} - \frac{1}{{K}_{h}} )}{\frac{{cos}^{2} θ}{{K}_{f}} + \frac{{sin}^{2} θ + ( h / l )}{{K}_{s}} + \frac{{cos}^{2} θ}{{K}_{h}}} = 1 \qquad ( 6 )$$

The value of the angle θ satisfying Equation 6 provides the Poisson's ratio symmetry.

The last geometrical parameter is determined by solving Equation 4 for the ligament thickness (t_{A}) of the anti-tetrachiral portion 12 (see Equation 21 and Equation 29).

Geometrical parameters and Young's modulus are normalized to be represented in the non-dimensional form as:

$$α = \frac{l}{m} \qquad ( 7 )$$

$$β = \frac{h}{m} \qquad ( 8 )$$

$${γ}_{N} = \frac{{t}_{N}}{m} \qquad ( 9 )$$

$${γ}_{A} = \frac{{t}_{A}}{m} \qquad ( 10 )$$

$$E = \frac{{E}_{2}}{{E}_{s}} \qquad ( 11 )$$

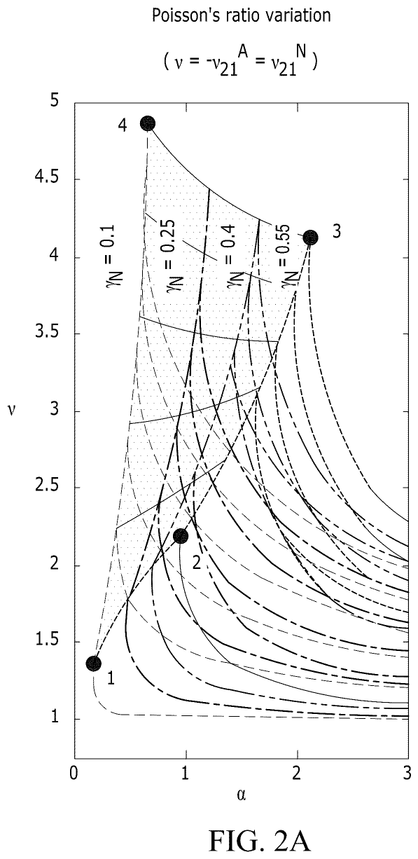

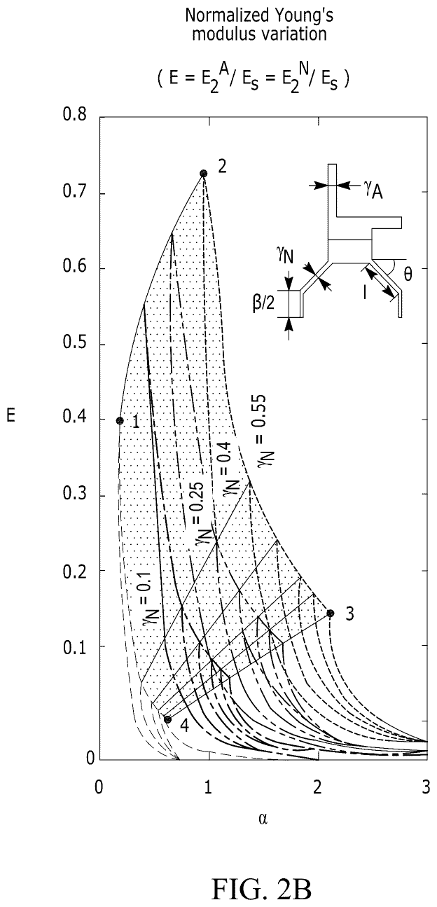

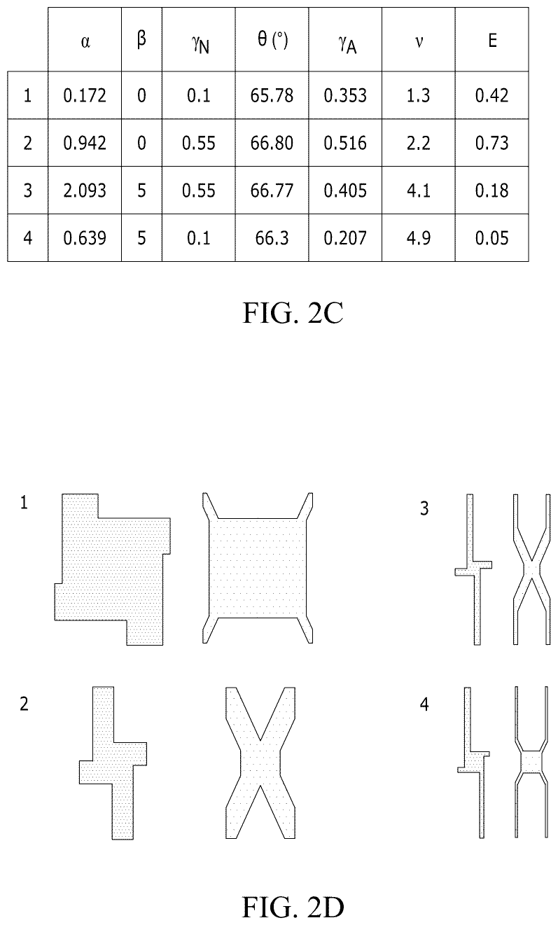

The Poisson's ratio (FIG. 2A) and normalized Young's modulus (FIG. 2B) were plotted with respect to a for multiple β and γ_{N} values (solid and dashed lines, respectively) to have a better understanding of nonlinear mechanical effects of predefined geometrical parameters. Lines in shaded areas defined the maximum value points of all curves that are theoretically possible. The geometric parameter combinations that defined the maxima of mechanical parameters (1, 2, 3 and 4) are tabulated in FIG. 2C, and schematically depicted in terms of corresponding lattice shapes in FIG. 2D.

The results demonstrates that vertical ligament 34 length (β) has greater influence on the Poisson's ratio (FIGS. 2A and 2C), while ligament thickness (γ_{N}) has greater influence on the Young's modulus (FIGS. 2B and 2C). Since increasing the vertical ligament 34 length (β) is not a theoretical limitation, no maximum value can be determined for Poisson's ratio mathematically. On the other hand, there are geometric constraints limiting increasing the Young's modulus. The ligament thickness (γ_{N}) can be increased until the ligament has the form of the nodal honeycomb geometry shown as model 2 (FIG. 2D). This model had the highest possible Young's modulus value of the AnA structures 10 evaluated (FIG. 2C).

### Analytical Model

Anti-Tetrachiral Unit Cells with Square Nodes

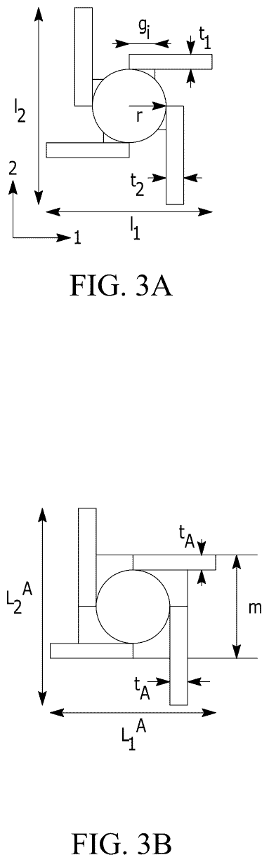

As background, an analytical model for mechanical properties of an anti-tetrachiral pattern with circular nodes was presented and later improved (see FIG. 3A). The original model calculates Poisson's ratio and Young's modulus values the anti-tetrachiral patterns, which have different thicknesses (t₁, t₂), lengths (l₁, l₂), and Young's moduli (Eₛ₁, Eₛ₂) of parent materials in horizontal and vertical ligaments. The improvement was made through adding the consideration of the ligament-node connection and its effect on the effective bending length of the ligaments. The reported analytical model is as follows:

$${v}_{i j} = - \frac{( 2 r + {t}_{j} ) {l}_{i}}{( 2 r + {t}_{i} ) {l}_{j}} \qquad ( 12 )$$

$${E}_{i} = \frac{1}{3 {( 2 r + {t}_{i} )}^{2}} \frac{{l}_{i}}{{l}_{j}} ( \frac{{E}_{s 1} {t}_{1}^{3}}{{l}_{1} - 2 {g}_{I}} + \frac{{E}_{s 2} {t}_{2}^{3}}{{l}_{2} - 2 {g}_{I}} ) \qquad ( 13 )$$

$$( i = 1 , 2 and j = 1 , 2 )$$

where r is the radius of the node, g_{I} is the length of the ligament-node connection.

In this example, we simplified the model by setting the ligament thicknesses the same and by using a single type of material.

$${t}_{A} = {t}_{1} = {t}_{2} \qquad ( 14 )$$

$${E}_{s} = {E}_{s 1} = {E}_{s 2} \qquad ( 15 )$$

Additionally, we used the considerations of the improved model to modify this analytical scheme and create a mechanical formulation for the pattern with square nodes having the edge length of m (FIG. 3B). The model with square nodes was derived by setting the ligament node length variable (g_{I}) in the analytical model to the accumulation of the radius of the circle (r) and the ligament thickness (t_{A}). With this adjustment, the ligament-node connection length (g_{I}) was assigned as the half-length of the square node width (m). The modified parameters were therefore as follows:

$${g}_{I} = r + {t}_{A} \qquad ( 16 )$$

$$m = 2 {g}_{I} \qquad ( 17 )$$

$${L}_{1}^{A} = {l}_{1} \qquad ( 18 )$$

$${L}_{2}^{A} = {l}_{2} \qquad ( 19 )$$

The purpose of changing the notation in the Equation 18 and Equation 19 was to eliminate the confusion with the inclined ligament of the honeycomb pattern which is addressed in the next section. After the simplifications and the modification, the resulting formulae for the construction appear as follows:

$${v}_{i j}^{A} = - \frac{{L}_{i}^{A}}{{L}_{j}^{A}} \qquad ( 20 )$$

$${E}_{i}^{A} = \frac{{E}_{s}^{A} {t}_{A}^{3}}{3 {( m - {t}_{A} )}^{2}} \frac{{L}_{i}^{A}}{{L}_{j}^{A}} ( \frac{1}{{L}_{1}^{A} - m} + \frac{1}{{L}_{2}^{A} - m} ) \qquad ( 21 )$$

$$( i = 1 , 2 and j = 1 , 2 )$$

### Nodal Honeycomb Unit Cell

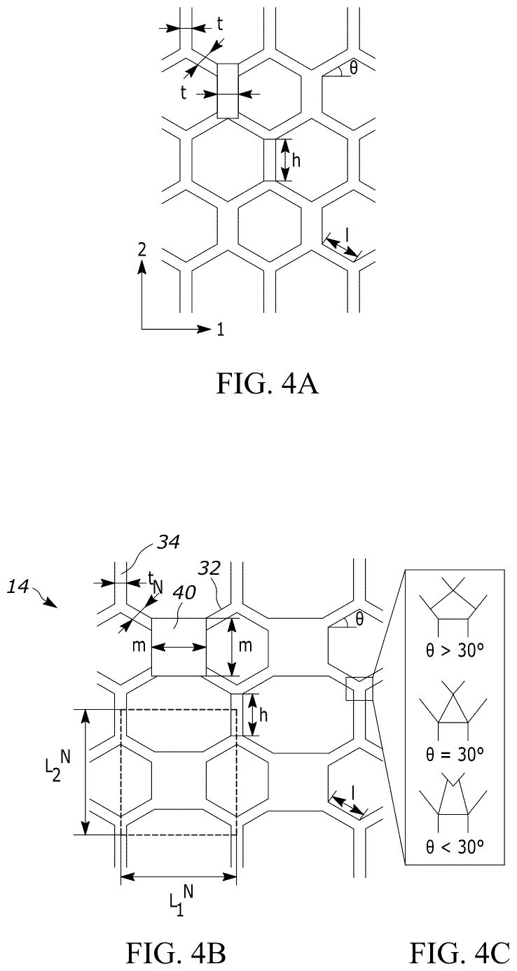

The nodal honeycomb portion of this example is a modified form of the conventional honeycomb shown in FIG. 4A. To this end, the nodal honeycomb portion was obtained by introducing finite sized, square-shaped nodes 40 into the conventional honeycomb lattice. More specifically, the nodes were generated by increasing the vertical ligament thickness (t) to derive the configuration shown in FIG. 4B. Analytical modeling of the nodal honeycomb capitalizes on existing mathematical principles for the conventional honeycomb of which deformation is characterized by flexure, stretching and hinging mechanisms. The combination of these three mechanisms with the added consideration of the effective ligament length has been previously shown to agree closely with FEA. We defined the mathematical model of the nodal honeycomb pattern 14 with the procedure that is similar to that followed by previous studies but with some deviations.

As one difference, instead of defining additional effective length parameters, we kept the ligament length and height (l and h, respectively) as effective lengths. We then designed suitable ligament intersection geometries shown in FIG. 4C and assumed to be mathematically rigid. In other words, the blackened ligament intersection in FIG. 4C were presumed rigid for modeling purposes. With that said, the horizontal (L₁) and vertical (L₂) size of the unit cell, as well as the ligament intersection geometry, varied depending on the angle θ.

The resulting mathematical model defining the Poisson's ratio and Young's modulus were expressed as follows (with the geometric variables being schematically depicted in FIGS. 4A-4C):

$$For 0 \leq θ \leq 30$$

$${L}_{1}^{N} = m + 2 l cos θ + {t}_{N} [ 1 - 2 sin θ ] \qquad ( 22 )$$

$${L}_{2}^{N} = m + 2 l sin θ + h \qquad ( 23 )$$

$$For 30 \leq θ \leq 90$$

$${L}_{1}^{N} = m + 2 l cos θ \qquad ( 24 )$$

$${L}_{2}^{N} = m + 2 l sin θ + h + {t}_{N} [ ( 2 sin θ - 1 ) tan θ ] \qquad ( 25 )$$

$${V}_{12}^{N} = \frac{{L}_{1}^{N}}{{L}_{2}^{N}} \frac{- cos θsinθ ( - \frac{1}{{K}_{f}} + \frac{1}{{K}_{s}} - \frac{1}{{K}_{h}} )}{\frac{{sin}^{2} θ}{{K}_{f}} + \frac{{cos}^{2} θ}{{K}_{s}} + \frac{{sin}^{2} θ}{{K}_{h}}} \qquad ( 26 )$$

$${V}_{21}^{N} = \frac{{L}_{2}^{N}}{{L}_{1}^{N}} \frac{- cos θsinθ ( - \frac{1}{{K}_{f}} + \frac{1}{{K}_{s}} - \frac{1}{{K}_{h}} )}{\frac{{cos}^{2} θ}{{K}_{f}} + \frac{{sin}^{2} θ + ( h / l )}{{K}_{s}} + \frac{{cos}^{2} θ}{{K}_{h}}} \qquad ( 27 )$$

$${E}_{1}^{N} = \frac{{L}_{1}^{N}}{{L}_{2}^{N}} \frac{1}{z} \frac{1}{\frac{{sin}^{2} θ}{{K}_{f}} + \frac{{cos}^{2} θ}{{K}_{s}} + \frac{{sin}^{2} θ}{{K}_{h}}} \qquad ( 28 )$$

$${E}_{2}^{N} = \frac{{L}_{2}^{N}}{{L}_{1}^{N}} \frac{1}{z} \frac{1}{\frac{{cos}^{2} θ}{{K}_{f}} + \frac{{sin}^{2} θ + ( h / l )}{{K}_{s}} + \frac{{cos}^{2} θ}{{K}_{h}}} \qquad ( 29 )$$

$${K}_{f} = \frac{{E}_{s} z {t}_{N}^{3}}{{l}^{3}} \qquad ( 30 )$$

$${K}_{s} = \frac{{E}_{s} z {t}_{N}}{l} \qquad ( 31 )$$

$${G}_{s} = \frac{{E}_{s}}{2 ( 1 + {ν}_{s} )} \qquad ( 32 )$$

$${K}_{h} = \frac{{G}_{s} z {t}_{N}}{l} \qquad ( 33 )$$

K_{f}, Kₛ and Kₕ represent stiffness values defining flexure, stretching, and hinging mechanisms respectively. Gₛ is the shear modulus and vs is the Poisson's ratio of the parent material. z is the unit pattern thickness.

### Example 2

### Tubular AnA Configurations as Expansional or Flexural Metastructures

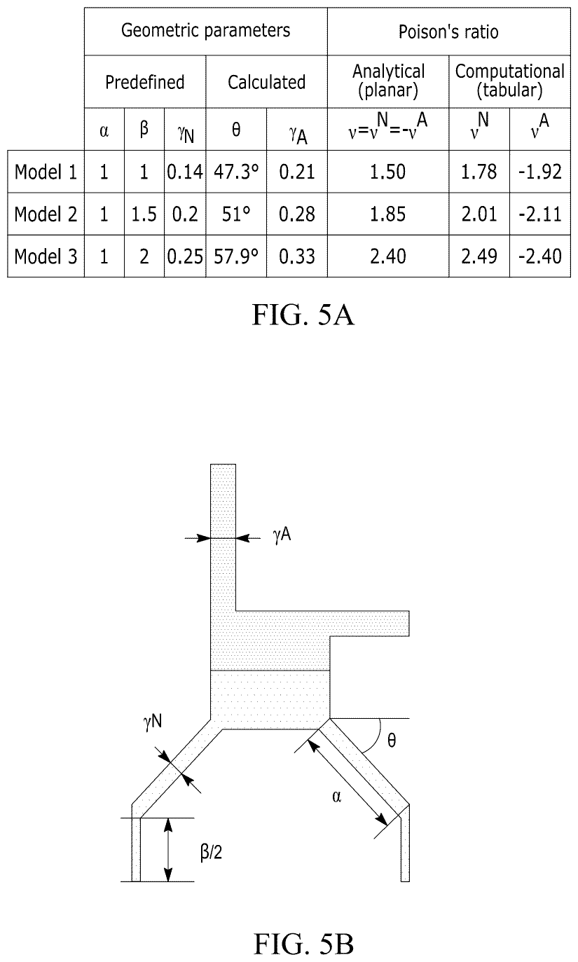

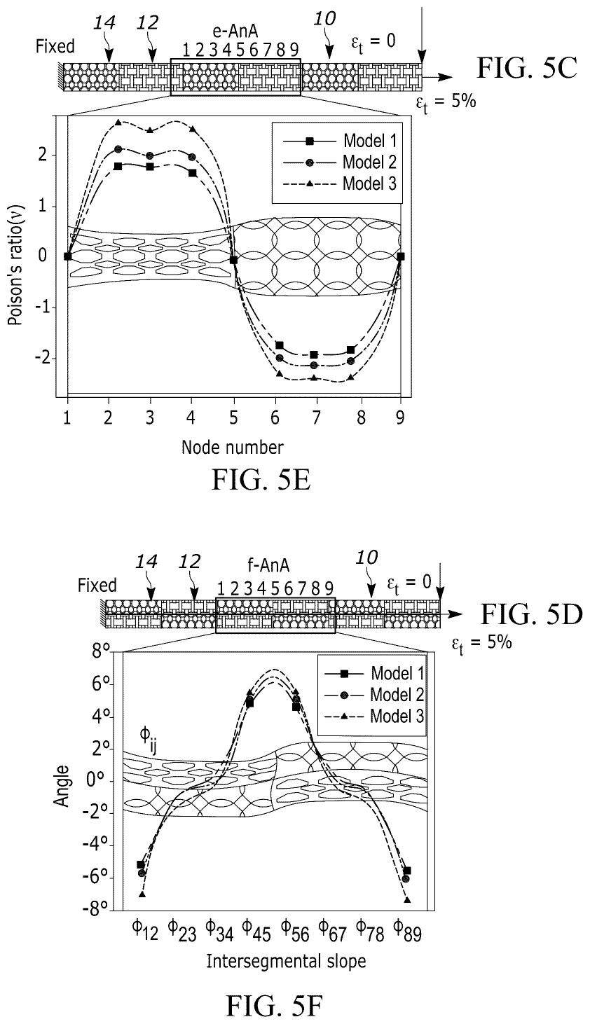

Auxetic and non-auxetic phases were assembled as tubular metastructure Models 1-3 (FIG. 5A) having interphase cell geometry (FIG. 5B) for the purpose of converging to metastructures 10 that display peristalsis-like (expansional) and snake-like (flexural) deformation patterns. Auxetic 12 and non-auxetic 14 phases of the metastructures 10 were designed parametrically and combined to create expansional AnA (e-AnA) tubular metastructures (FIG. 5C) and flexural AnA (f-AnA) tubular metastructures (FIG. 5D) made of thermoplastic polyurethane (TPU). The middle phases of uniaxially deformed tubes were analyzed to quantify the deformation of e-AnAs (FIG. 5E) and f-AnAs (FIG. 5F) tubular metastructures and their variations between different lattice models. To this end, we visualized the deformation patterns of tubular arrangements of AnA metastructures using FEA (FFEPlus solver, Static mode, SolidWorks® 2019) and analyzed the three models (Models 1-3 from FIG. 5A).

Geometric models were first designed in SolidWorks as flat, and then rolled into tubes using the flex feature. The tubular metastructures 10 had a fully fixed boundary condition on one end (left as shown in FIGS. 5C-5D). The other end (right as shown) underwent a prescribed displacement resulting in 5% elongation along the longitudinal axis (8=5%). Transverse movement on the longitudinally deforming end was confined (&t=0) (see FIGS. 5C and 5D).

The tubular metastructures 10 were meshed with a high quality, solid mesh of tetrahedral elements with 5% tolerance. Although TPU is a non-linear material, we defined it as a linear elastic material with a Young's modulus value of 26 MPa because the applied strain of 5% is well within the yield strain of TPU at 55%. Both the e-AnA and f-AnA tubular metastructures 10 were designed as six longitudinally connected segments with a pattern thickness of 2 mm.

The e-AnA tubular metastructure 10 of FIGS. 5C and 5E resulted from alternating repetitions of the non-auxetic 14 nodal honeycomb segments and the auxetic anti-tetrachiral segments 12. Each segment consisted of four unit-cells (4×L₂) along the longer axis of the tubular metastructure 10, and eight units along its circumference (8×L₁). FEA simulation confirmed the expected peristalsis-like deformation pattern where segments alternatingly expanded or contracted under uniaxial extension (FIG. 5E).

The f-AnA tubular metastructure 10 of FIGS. 5D and 5F was obtained by arranging the auxetic and non-auxetic phases 12, 14 in a checkerboard pattern six segments long (FIG. 5D). One half of each segment was auxetic 12 with four unit cells along the length (4×L₂) and four unit cells along the arc (4×L₁). The other half of each segment was non-auxetic 14 with the same repeat number of unit cells along its length and arc. In consecutive segments in both the length and arc directions, the auxetic phase 12 of one segment grafted with the non-auxetic phase 14 of the next segment. In line with the postulation, f-AnA tubular metastructures 10 underwent periodic bending under uniaxial deformation.

The data representing displacement in the transversal plane was collected from the central points of the nodes at only the middle sections of the tubular metastructures 10 in order to minimize the boundary effects. The amount of nodal cross-section expansion was divided by the diameter of the undeformed tube 10 to calculate transverse strains. Poisson's ratios (ν) were calculated as the negative value of the ratio of strains (ε) in the transverse and longitudinal axes. Poisson's ratio (ν) variations of the AnA tubular metastructure 10 were determined by repeating the calculation for each nodal cross-section (FIG. 5E).

Although shape morphing of e-AnA tubular metastructure 10 can be expressed as Poisson's ratio (ν) variation because the material deformation under loading is concentric throughout the longitudinal axis, the deformation characteristic of the f-AnA tubular metastructure 10 is not compatible for this quantification with Poisson's ratio (ν). This is because both expansion and contraction modes are present in each cross-section of the f-AnA tubular metastructure 10. Consequently, deformation of the f-AnA tubular metastructure 10 was expressed as the intersegmental slope (FIG. 5F), which is the angle Φ between the central line in the deformed structure and the longitudinal axes of the undeformed structure.

FEA results demonstrated that the geometrical parameters obtained from the analytical approach provided approximately equal (within 8%) amplitudes of lateral expansion and contraction in the auxetic and non-auxetic phases 12, 14 of the e-AnA tubular metastructure 10. The difference between analytically and computationally determined Poisson's ratios (ν) of e-AnA tubular metastructures 10 decreased in high Poisson's ratio (ν) values. While previous metamaterial computational results have been improved to match computational results and analytical predictions, here we observed greater differences because of the periodically changing boundary conditions between sequential phases.

Furthermore, since the lattices were designed flat and then wrapped into a tubular shape, the effective lengths of the ligaments in the radial direction are related to chord lengths, which are less than the designed lengths along the circular arc. Consequently, wrapping the flat lattice into tubes, which increases the aspect ratio and thus the Poisson's ratio. Therefore, in Model 3, which has the longest distance between boundaries, the Poisson's ratio's measured at the center approach the analytical result. In the e-AnA and f-AnA tubular metastructures 10, the amplitude of expansion/contraction and bending (respectively) can be controlled by changing the Poisson's ratio through adjustment of the geometric properties of unit lattices. In other words, a greater amount of expansion-contraction and bending is observed with increasing Poisson's ratio (ν).

### Example 3

### Manufacturing of AnA Tubes

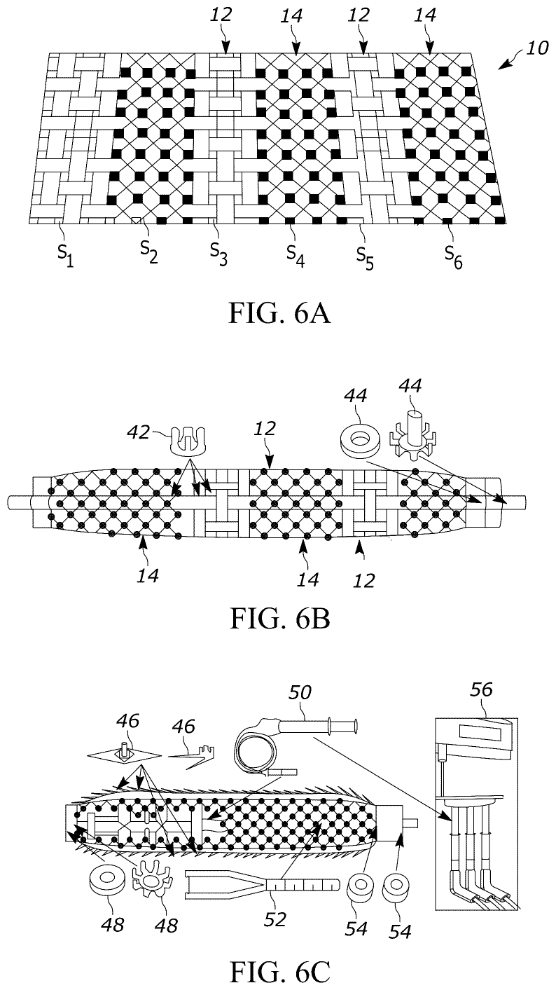

In this study, AnA structure 10 models were fabricated in planar form (FIG. 6A) and then rolled into tubes (FIG. 6B). Instead of using one of the models performed in the computational study, a smaller AnA model was designed for this example because of the limited build area of 3D-printer used (Ultimaker S3, Ultimaker, Zaltbommel, the Netherlands). The AnA model, which was built with Ultimaker TPU 95A, was designed with six longitudinal sections (indicated at S₁, S₂, . . . . S₆ in FIG. 6A), each of which is of three unit/cell long and has eight units along the circumference.

Once the planar structure 10 was rolled into a tube, mechanical snap connectors 42 (FIG. 6B) were used to join and hold the nodes 24, 40 on the long edges of the structure to form a seam. The connectors 42 were designed and 3D-printed with a hard material (Ultimaker Tough PLA) to maintain the structure 10 in a cylindrical shape. End connectors 44 were designed to close one end of the tubular structure 10 and maintain the nodes 24, 40 at that end of the tube in a circumferential pattern. A fixing member 48 is provided to close the other end of the tubular structure 10 and maintain the nodes 24, 40 at that end of the tube in a circumferential pattern.

Snap-on triangular scales or extensions 46 (Ulimaker Tough PLA) were provided along the length of the tubular structure 10 to attain frictional anisotropy with the terrain. To this end, the extensions 46 all extended in the same longitudinal direction. The extensions 46 were 3D-printed as flat and one of the extensions was folded to obtain unidirectional movement and placed both along the seam as well as on some of the lateral nodes 24, 40.

The tubular structure 10 was actuatable by an actuation member 50 connected to the fixing member 48. In this instance, the actuation member 50 included a larger water-filled syringe, a smaller syringe, and a tube connecting the two. A screwed rod 52 was connected to the end connector 44 and by a pair of end bolts 54. The small syringe of the actuation member 50 was placed inside the tubular structure 10 and its movement transferred to the front of the tubular structure 10 by the screwed rod 52 and end bolts 54. The larger syringe of the actuation member 50 was placed in a mechanical test machine 56 which was used converted to a syringe pump.

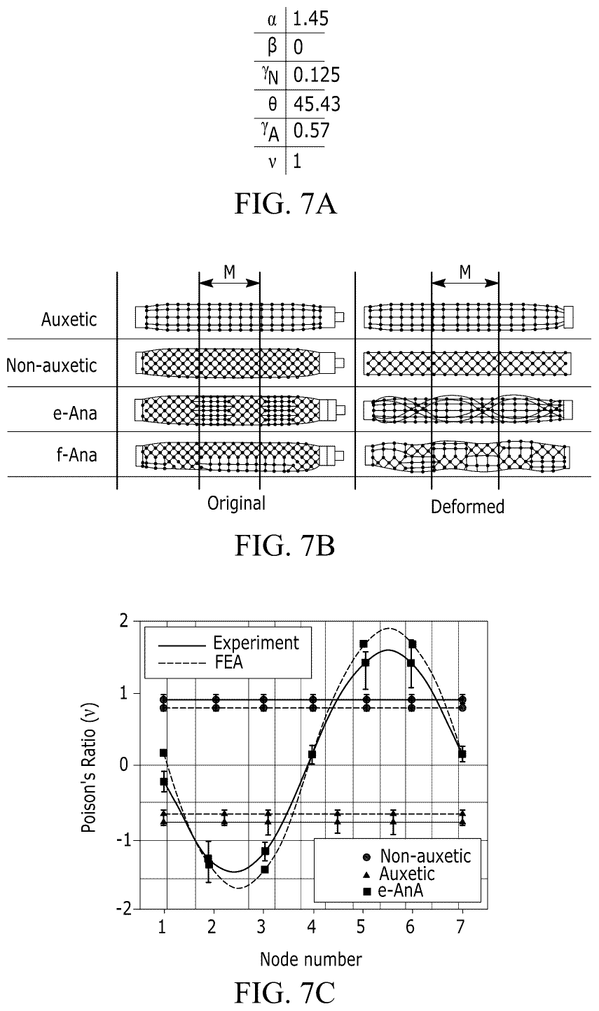

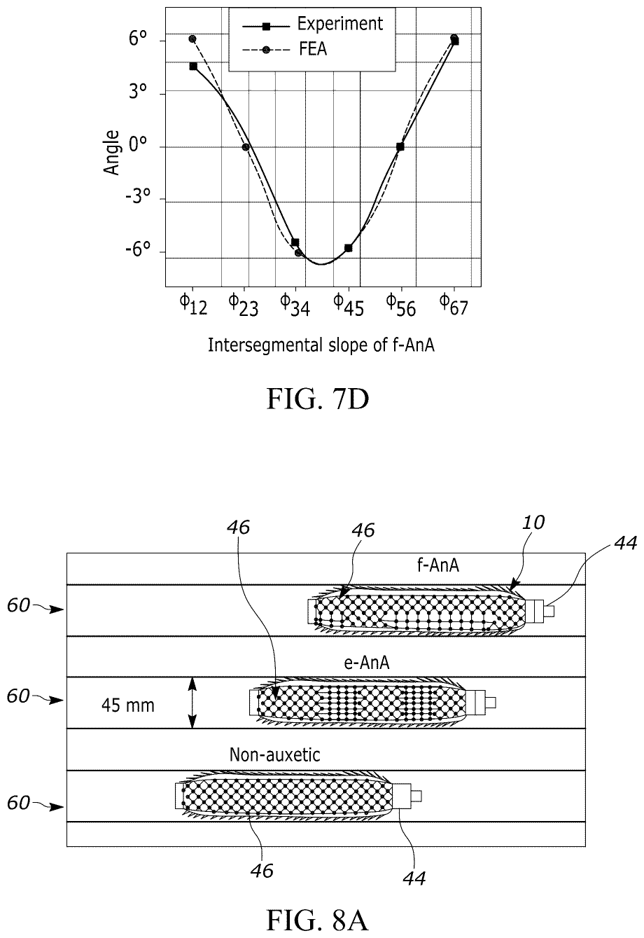

The design procedure of the fabricated model is different from analytical and computational models. Consequently, we conducted mechanical tests on multiple tubular structure 10 samples having different phases (auxetic, non-auxetic, e-AnA, f-Ana) to investigate the effects from design differences. The lattices were designed with the same geometrical parameters (FIG. 7A) and 3D printed with Ultimaker TPU. In particular, we prepared three sets of auxetic, non-auxetic, e-AnA and f-AnA samples 10, and placed markers M on the lateral nodes 24, 40 of the two phases, located in the middle of the tubes (FIG. 7B).

### Testing of AnA Tubes

Samples were placed on a mechanical test machine 56 (Test Resources 800LE3-2; Test Resources Inc., MN) and a camera was placed 1.5 m away from the samples. The end connectors 44 enabled clamps on the test machine 56 to grip the samples.

Rolling the flatly fabricated metastructures 10 into tubes caused a lateral contraction. This contraction results in an axial elongation for non-auxetic lattices and an axial shortening for auxetic lattices. To eliminate this effect, and to match the length of tubes with the configuration in their FEA counterpart, the in-plane lengths of the structures 10 were taken as reference and structures were pre-loaded to keep them at the same lengths (“Original” in FIG. 7B) before loading (“Deformed” in FIG. 7B).

Photographs were taken at the original designed position (“Original” in FIG. 7B), and after being deformed in tension to a level of ˜5% strain (“Deformed” in FIG. 7B). Marker M positions were digitized by using an image analysis software (NIH, ImageJ). Poisson's ratio for concentrically deforming lattices (FIG. 7C) and intersegmental slope for f-AnA (FIG. 7D) were calculated by using the collected marker data via the same method with computational analysis.

### Results

By generating approximately equal amount of shape changing with a 6% difference in auxetic and non-auxetic phases of e-AnA, the experiment fulfilled the aim to generate close magnitude of expansion-contraction in both lattices (see FIG. 7C). It was also observed that the design differences cause a negligible effect in bending of computational and experimental f-AnA models as shown in FIG. 7D (in which the error bars stand for standard deviation).

While others have shown the accuracy of FEA in modeling deformations for honeycomb and anti-tetrachiral lattices, to our knowledge, the effects of wrapping the lattice into a tube has not been previously studied. With this in mind, our experimental Poisson's ratio was 87% of FEA results for non-auxetic and 94% for auxetic. The agreement suggests that flat lattice structures can be conveniently printed flat and then folded into 3D shapes with desired Poisson's ratios. This is a promising alternative to the challenges of directly 3D-printing lattices in cylindrical form without changing deformation properties.

Furthermore, we validated the modularity of these spatially matched lattice sections by conducting these same experiments with e-AnA and f-AnA configurations. Experimental predictions were within 88% of FEA for e-AnA and 96% for f-AnA. Taken together, the modularity, analytical approximations, and ease of fabrication make these AnAs accessible for broader applications, such as new soft robotic devices. Furthermore, the agreement between experimental and computational analyses implies that different shape-morphing lattice combinations can be surveyed computationally to identify suitable geometries for a defined outcome, to reduce the design space prior to the implementation of time-consuming manufacturing.

This study showed that the nodes can be used to connect surfaces to each other (e.g., here to form a tube). The flat lattices can be stitched together like squares in a quilt and then deformed into 3D spaces. This makes AnA metamaterials more accessible to potential users-either with novel materials or novel applications. Different or similar phases can be snapped together using the connectors 1, 2 with almost invisible seams. More importantly, the seams have negligible effect on overall behavior, as we showed with our experimental results. Custom skins with auxetic or non-auxetic “pixels” that open, tighten or loosen at particular spots could be valuable in mechanism design, wearables, or packaging.

### Example 4

Motile Robots with AnA Skins

The ability of AnAs to generate multiple deformation modes may be highly promising for use in many applications. Especially, having dynamic shape-morphing characteristics may improve compliance to the environment, speed performance, and the ability to move in confined spaces. Moreover, the analytical design concept which enables tuning of the Poisson's ratio may help optimize the shape-changing amplitudes for specific spatial requirements. Poisson's ratio is especially critical for worm-like crawling in an idealized model—the greater the Poisson's ratio, the more efficient the robot.

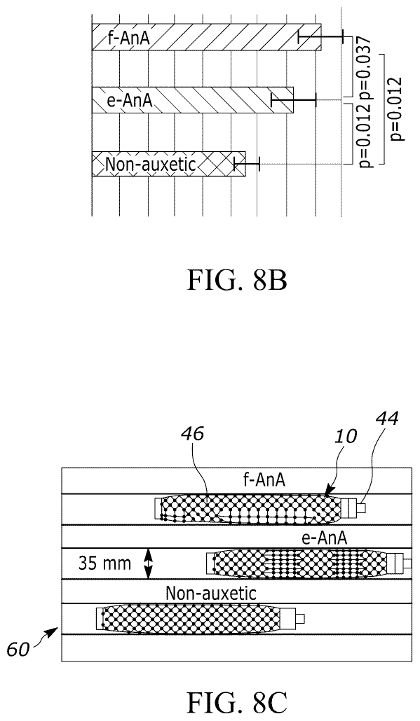

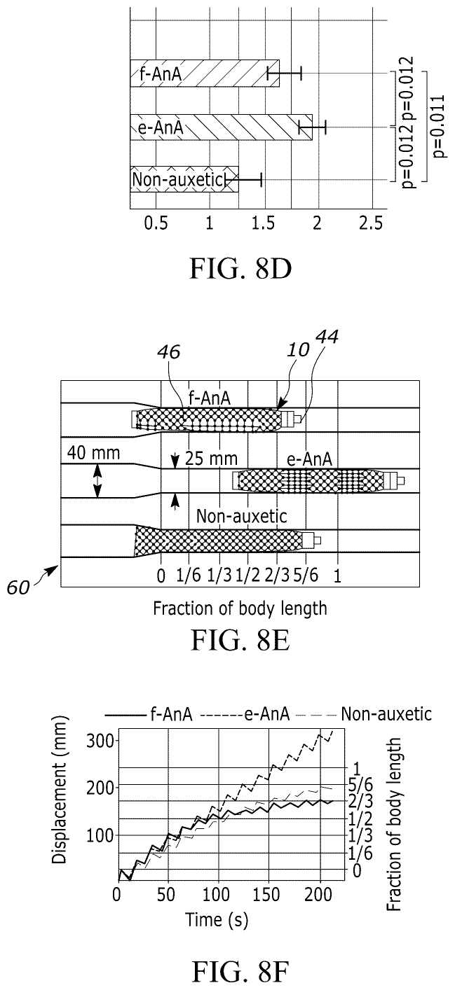

We have previously shown how mechanisms with an effective Poisson's ratio of 1.5 enable segments to both advance and anchor. Others have shown that auxetic and non-auxetic lattices can be added to inchworm robots to enable alternating engagement and detachment. Here we present how using AnA lattice skins can improve the motility of soft robots in channels 60 (FIG. 8A). As an illustration of the capabilities of the AnAs, we focused on channels 60 with varying widths to simulate different types of tunnels or burrows where soft animals are particularly adept in navigating. The channels 60 were covered with 5 mm thick foam sheets to improve the frictional anisotropy.

The motile structures 10 to be tested were created with the same AnA structures and isotropically deforming non-auxetic lattice designed for the mechanical testing (see FIG. 6B). To this end, the aforementioned triangular scales 46 provided on the lateral nodes 24, 40 of the structure 10 are akin to parapodia on some worms or scales on snake-inspired kirigami. These scales 46, in combination with the foam covering the channel 60, provided frictional anisotropy. That said, three structures 10 (f-AnA, e-AnA, Non-auxetic) were each placed in one of the channels 60 with the scales 46 pointing away (left as shown) from the direction of travel (right as shown).

Testing in this example was similar to that shown and described in FIGS. 6B-6C. To this end, the fixing member 48 was positioned within each structure 10. To move the motile structures 10, a hydraulic system was prepared to actuate both the AnAs 12 and non-auxetic 14 lattice simultaneously. This system consists of the mechanical test machine 56 and three actuation members 50 (one for each structure 10) coupled to the respective end bolts 54 on each structure. Three large syringes (60 ml) of the actuation member 50 were placed on the mechanical test machine 56. The small syringes (10 ml) of the actuation member 50 were assembled in the lattices between the ends of the structure 10 to generate uniaxial loading. The test machine 56 was programmed to generate periodic contraction and tension in a 12 mm range with the feed rate of 3 mm/s, and 1 second of wait at extremum points. All three structures 10 were tested with the same testing machine 56 in parallel with one another, i.e., at the same time.

The lattices were tested for three test-cases: 1) 45 mm channel width (FIG. 8A), 2) 35 mm channel width (FIGS. 8C-8D) and 3) narrowing channel (from 40 mm to 25 mm) (FIGS. 8E-8F). The movements of robots were recorded in five replicates for each case from the top view with a camera. Markers were placed at the tip of the robot structures 10 and data were collected. Recorded videos were analyzed by a commercial software (Photron FASTCAM Viewer). Significant differences between the velocities of the motile structures 10 were tested by using a non-parametric Kruskal-Wallis test (P-values of 0.003 for 45 mm wide channel and 0.002 for 35 mm wide channel), and post hoc pairwise comparisons were performed by using Mann-Whitney U-test (N=5). Significance was reported at the level of p<0.05 and error bars shown standard deviation.

Results showed that when the channel width is 1.3 times larger than the diameter of the metamaterial tube (45 mm), the f-AnA metamaterial structure moves 12% faster than e-AnA and 41% faster than isotropic non-auxetic structures (FIGS. 8A and 8B). Consequently, in the 45 mm channel 60 the f-AnA metamaterial structure has the best motility and overall speed.

On the other hand, the e-AnA structure showed better motility (19% faster than f-AnA and 53% faster than non-auxetic lattice) in smaller channels with width (35 mm) approximately equal to the metamaterial tube diameter (32 mm not including scales) (FIGS. 8C and 8D). In both types of channels, the non-auxetic robot was the slowest. These results emphasize that periodic shape-morphing of AnA may enable soft robots to test strain patterns observed in animals which can improve the performance of biomimetic soft robots in the future.

Turning to FIGS. 8E-8F, AnA tubes can address a “bottleneck challenge”, in which the channel 60 gets narrower than the metamaterial tube radius along the path of motion. We have previously shown such a challenge can be addressed by complex control of different segments. Here we showed that the e-AnA configuration can smoothly transition in response to the channel width without changing velocity passively or requiring active control.

With this in mind, and turning specifically to FIG. 8E, we started all three configurations in a channel 60 with an initial, intermediate width (40 mm), such that all three tubular structures 10 can locomote. Propelled by contact with the channel 60 walls, all three can enter the narrowing. However, only the e-AnA structure continues to travel within the narrowing, while the f-AnA and non-auxetic structures become jammed and cannot progress when the ˜4 section-long part remains in the wider channel.

We note that the starting position of the tubes 10 were such that they reached the beginning of the narrowing (Line 0) after one cycle. These demarcation lines represent the displacement of white markers (the front end of the lattice), which are numbered with the fraction of the length of the tube in the narrow channel. The original length of the tubes was 220 mm.

The success of e-AnAs is due to the fact that the metamaterial phases within the narrowing continue to expand and contract, gaining purchase via the frictional anisotropy. In contrast, f-AnA does not change its diameter as much (total Poisson's ratio=0). While the isotropic structure can expand and contract (total Poisson's ratio=+0.9), it can do so only in unison along its length. Thus, in highly confined spaces, structures having a Poisson's ratio that varies along its length (such as between −1.4 and +1.4 for e-AnA) is uniquely valuable for motility.

Here we have shown the advantages of combining auxetic and non-auxetic patterns in a grid-like configuration for soft robotics, namely, AnAs have better motility than isotropic materials in confined channels. Specifically, e-AnA embodies in one matrix the balance of expansion and contraction that we previously hypothesized is required by a series of peristaltic muscle activations. As a result, e-AnA deforms more evenly in confined tubes. In contrast, isotropic lattices demonstrate localized deformation (aka jamming), and therefore are less apt to traversing confined spaces. These materials enable scalable solutions to established locomotion challenges such as transitioning through a bottleneck that would otherwise limit worm-like and snake-like soft robots. Such robots can have application, for example, in accessing buried infrastructure, medical stents and implants, etc. These simple lattice frameworks can also facilitate broader use of bending actuator concepts and other customized metamaterials yet to be conceived.

### Example 5

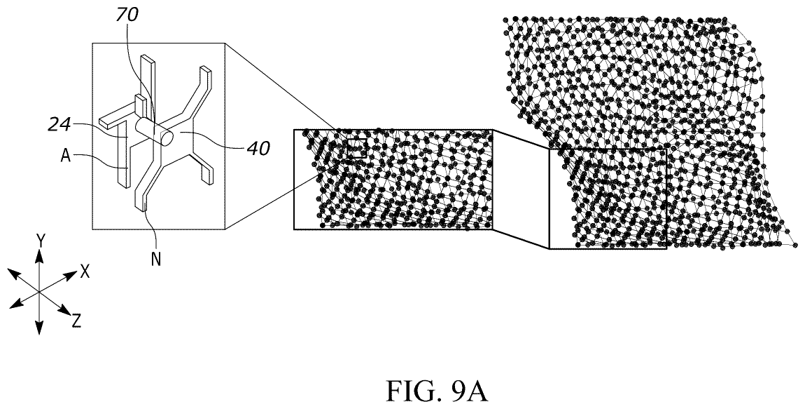

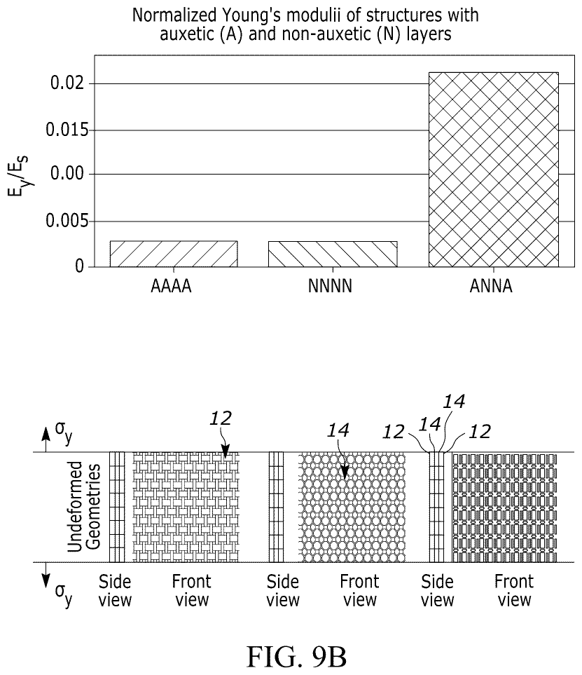

In this example shown in FIGS. 9A-9B, nodal auxetic (A) and non-auxetic (N) lattices were combined in out-of-plane stacking configurations with the geometrical parameters given as Model 1 in FIG. 3A and investigated via FEA. Stacking the AnA lattices in this matter enabled out-of-plane deformation or stiffening. This is shown computationally in FIG. 9A.

If an auxetic layer and a non-auxetic layer were stacked in the z-direction and connected with pins 70 at the nodes 24, 40, out of plane (z-direction) protrusions emerge for in-plane axial loading. In particular, results show that z-AnA (FIG. 9A) takes a wavelike shape bulging at the auxetic side under uniaxial tension.

In contrast, symmetrically stacked auxetic and non-auxetic structures (A-N-N-A or N-A-A-N) produce an order of magnitude higher longitudinal stiffness than combinations of single-type isotropic lattices (A-A-A-A or N-N-N-N) (FIG. 9B). In other words, simulations demonstrate that symmetrically stacked auxetic and non-auxetic layers have higher (for this case 7.4 times higher) Young's modulus than monophasic structures (FIG. 9B). This outcome supports the conclusions of a previous computational study in which auxetic and non-auxetic combinations were modeled as continuum materials.

The source of this increasing stiffness originates from the opposite flow of metamaterials in neighboring layers under loading. Being able to induce different stiffnesses and 3D deformations enables finer biomimicry at scales smaller than actuators, for example to create load-sensitive fingerprint ridges, to define creases for folding, or to dynamically vary friction of feet.

### Example 6

In this study, we investigated the mechanical behavior of a novel 2D metamaterial concept, which is serially stacked auxetic-non-auxetic combinations. Anti-tetrachiral (ATC) model has been preferred as auxetic part and a non-auxetic pattern which is compatible for combining with chiral patterns. As a result of this study, we are presenting an analytical design protocol to determine unit dimensions for exact opposite Poisson's ratio and equal Young's modulus in auxetics and non-auxetics under the constraint of equal node spacing in the same axis.

### Materials and Methods

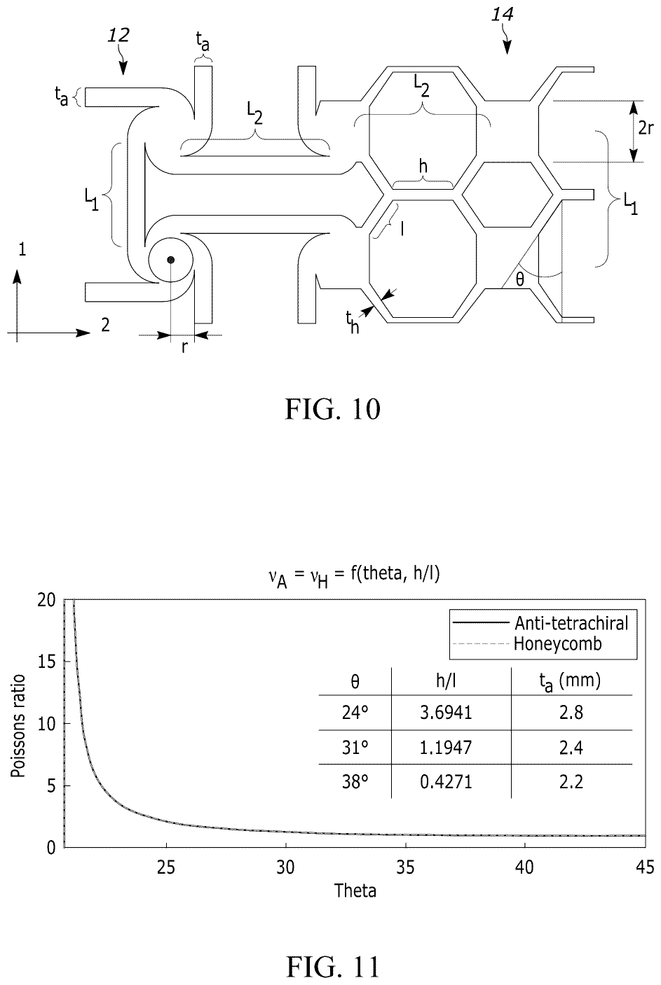

In previous studies, re-entrant auxetic structures are indicated as a derivation on honeycomb structures. Contrary to re-entrant, chiral structures which consist of nodes and ligaments do not have any relatable non-auxetic structure. As a result of this need, we designed a honeycomb-inspired chiral structure (HC) (see FIG. 10) for which the same mathematical equations of conventional honeycomb structures may be used.

Equal node spacing is the first condition we desired. The reason for this is to make the geometrical parameters relatable:

$${L}_{1} = 2 l c o s θ + 2 r ; {L}_{2} = h + 2 l s i n θ + 2 r \qquad ( 34 )$$

For the case of serial stacking meta-combination requires a constant Poisson's ratio. The purpose behind this is to provide the exact opposite transverse deformation in auxetic and non-auxetic portions 12, 14 under longitudinal loading. Since exact negative deformation depends on the same amount of loading the Young's modulus (E) in every cross-section of the structure had to be equal as well so that the deformation applied could be equally distributed to the auxetic and non-auxetic portions 12, 14.

Upon these considerations, previously reported mathematical models of honeycomb and ATC structures have been utilized with relatable geometrical parameters (eq. 35). Independent variables, θ and h/l were correlated to the condition of equal Poisson's ratio (eq. 36). Substitution of corresponding geometrical parameters into the Young's modulus formulas (eq. 37) enables to determine the required ligament thickness of ATC pattern, which is a fundamental parameter for stiffness.

$${v}_{21}^{A} = - \frac{{l}_{2}}{{l}_{1}} = \frac{h / l + 2 r / l + 2 sin θ}{2 r / l + 2 cos θ} ; {v}_{21}^{H} = \frac{( ( h / l ) + sin θ ) sin θ}{{cos}^{2} θ} \qquad ( 35 )$$

$$- {v}_{21}^{N} = {v}_{21}^{H} \to ( h / l ) = f ( θ ) \qquad ( 36 )$$

$${E}_{2}^{A} = \frac{{E}_{s} {t}_{A}^{3}}{3 {( 2 r + {t}_{A} )}^{2}} ( \frac{{L}_{1} + {L}_{2}}{{L}_{1}^{2}} ) = \frac{{E}_{s} {t}_{A}^{3}}{3 {( 2 r + {t}_{A} )}^{2}} ( \frac{h / l + 4 r / l + 2 sin θ + 2 cos θ}{l * {( 2 r / l + 2 cos θ )}^{2}} )$$

$${E}_{2}^{H} = \frac{{E}_{s} {t}_{h}^{3} [ ( h / l ) + sin θ ]}{{l}^{3} {cos}^{3} θ} \qquad ( 37 )$$

### Results

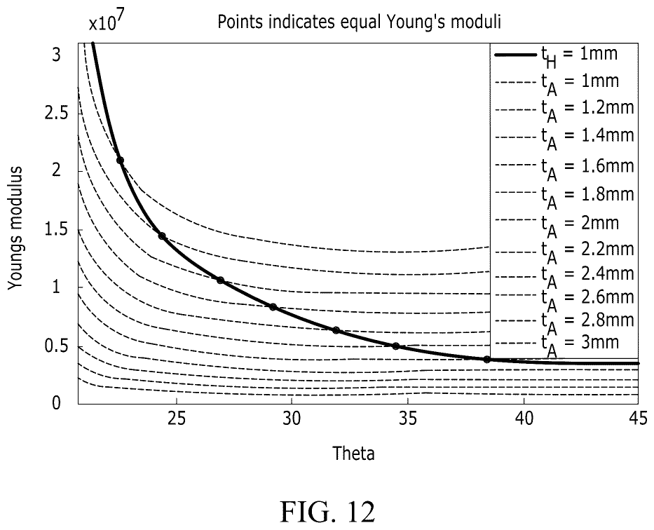

The results shown in FIGS. 11 and 12 demonstrate that the data generated by the analytical model was valid between the angle θ values of 20.7° and 45° for equal Poisson's ratio and Young's modulus in ATC and HC structures (with r=3 mm, l=7 mm, tₕ=1 mm). The data that is sourcing from outside of this interval was found practically incompatible for designs. The results also illustrated that h/l and ligament thickness tₐ decrease to maintain the equality of ν₂₁ and E₂ as values for the angle θ increased.

### Example 7

In this study we proposed a metamaterial design procedure for personalized-stents consisting of auxetic (anti-tetrachiral) and non-auxetic (nodal honeycomb) pattern phases. The developed algorithm analyzed the geometrical configuration of the presumed canal receiving the stent, discretized the surface defining the canal based on local shape formation, and generated the required pattern type and Poisson's ratio for each section (longitudinal and/or circumferential) of the stent.

### Methods

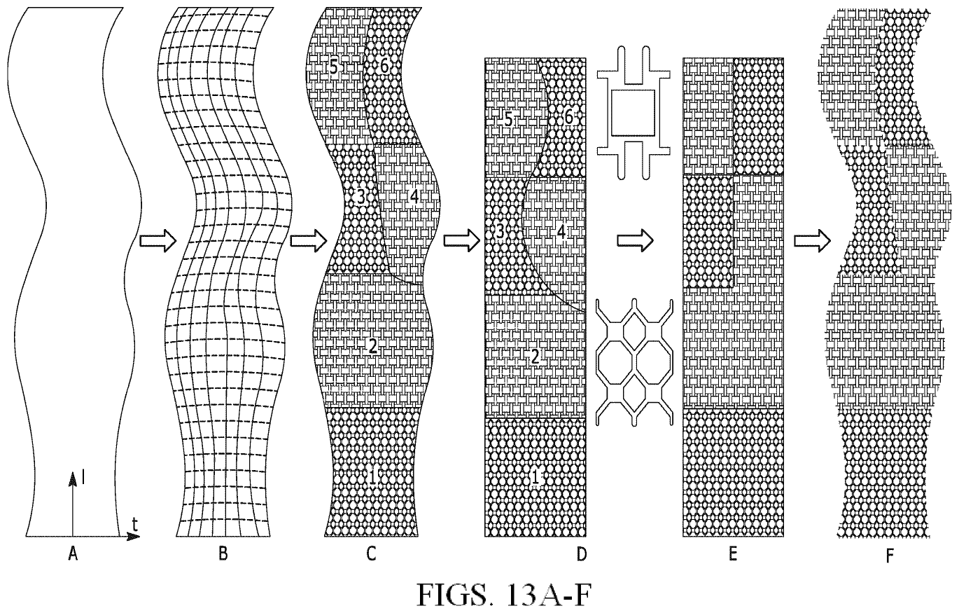

Our design methodology used the anatomical geometry as the deployed, deformed shape of the stent based on personalized imaging as a starting point. The approach then generated the uniform, deformed tubular structure as a shell body (FIG. 13A). Sample input canal geometries with expanding, contracting and bending sections were designed as surfaces of the shell body. The shell body geometry of FIG. 13A was then meshed with 4-node rectangular elements in Matlab (FIG. 13B).

An undeformed version of the shell body was generated (FIG. 13D) by fixing the bottom end of the deformed body and inputting the longitudinal strain on the body. In particular, the undeformed diameter of the body was set as equal to the bottom end of the input geometry. The length was determined by reducing the length of the deformed geometry by the assumed applied longitudinal strain for deployment (10%). Coordinates for each of the deformed and undeformed body were then generated. The developed algorithm then calculated the distance of each node from the central axis of the deformed body and compared that distance with the undeformed body diameter.

These comparisons dictated which sections of the body expanded (indicated at 2, 4, 5 in FIGS. 13C-13D) and which sections contracted (indicated at 1, 3, 6 in FIGS. 13C-13D). Colors (red for expansion, blue for contraction, and green for neutral) were then mapped onto both the deformed (FIG. 13C) and undeformed (FIG. 13D) bodies. More specifically, the color map was transferred from the deformed body to the undeformed body so as to function as a guide to build the stent in the next steps. At the same time, the maximum and minimum node distances from the central axis were used to calculate the Poisson's ratio of each section. The nodes of which distance from the central axis were equal to the radius of the undeformed geometry were used to determine auxetic and non-auxetic phase boundaries.

It will be appreciated that since the geometry of the shell bodies with quadrilateral mesh elements are used as the input, a 2D FEA code can be utilized (see FIG. 14) to define the deformation behavior of each mesh element. This would allow for the creation of a computationally reversible material model for modeling shape transformation.

In order to combine different patterns, their unit structures must be relatable and have mutual geometrical features. Nodal honeycomb pattern was developed with the addition of square node to conventional honeycomb. This addition enabled auxetic anti-tetrachiral pattern to be combined with a non-auxetic pattern. For the geometrical integration the unit sizes were set as the same. The geometrical parameters of patterns with required Poisson's ratios were created by using the analytical models defining in plane mechanical characteristics. Since we programed the metastructure by tuning the Poisson's ratio (xv₁=yv₂=zv₃= . . . ), the Young's modulus (E) of each section was set to the same value (Eᵢ=Eⱼ, i,j=1,2,3, . . . ).

The unit geometries were designed according to the output of the geometrical analysis algorithm and results of the mathematical models providing mechanical integration. The stent (FIG. 13E) was built by assembling designed pattern pieces according to the color map on the undeformed body (FIG. 13D). The resulting stent model was then tested via finite element analysis (SolidWorks 2019) by loading the model (see FIG. 13F) with the same pre-defined longitudinal strain value (10%) to compare with the initial canal geometry.

### Results

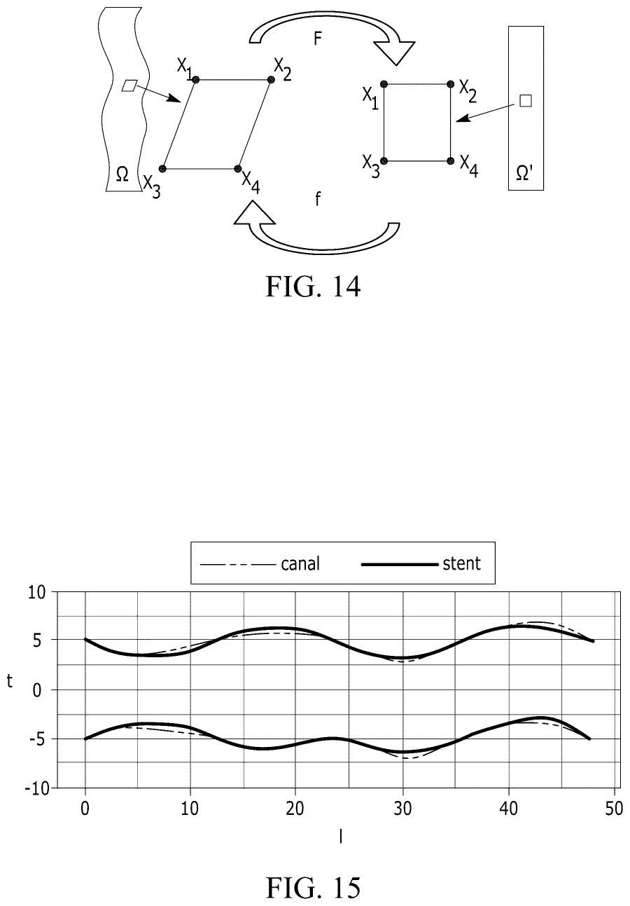

Data collected from edges of the deformed geometries (canal and stent) in It-plane were plotted (FIG. 15). The result shows a successful coherency with a maximum of 11% error. The error may be eliminated by creating a Poisson's ratio gradient within each section.

This study proposed a novel perspective for the stent technology by creating different shape-morphing characteristics in different regions of the structure, which takes the geometric form of the targeted environment. This concept may be enriched with addition on other patterns. The next stage of the process will involve fabrication of patterns emerging for this design process using 3D printing and assessing their conformation to anatomical features that are derived from MR images.

A primary contribution of this work is the rational design of AnAs-metamaterials with combined auxetic and non-auxetic phases in a single lattice. Prior to this work, auxetic and non-auxetic combinations were limited to conventional honeycomb and re-entrant lattices. Our work enables the use of different auxetic patterns (specifically anti-tetrachiral which had no previously established application) and introduces the first non-auxetic nodal pattern (nodal honeycomb). Modular grids were created with nodal honeycomb and anti-tetrachiral unit cells (FIG. 1).

The unit cell geometry was designed with our analytical relationships for symmetric deformation (−v^{A}=v^{N}) and matching mechanical (E^{A}=E^{N}) properties (FIGS. 2A-2B). AnA metamaterials with periodic Poisson's ratio variation were demonstrated computationally (FIG. 5A) and experimentally (FIG. 7C). AnAs can generate spatial patterns of expansion-contraction (e-AnA) or flexure (f-AnA), which result in locomotion under periodic uniaxial loading (FIG. 8F).

It will be appreciated that the concepts and approach shown and described herein can also be used to create multi-stiffness materials (E^{A}≠E^{N}) for layer jamming, smoothly varying Poisson's ratio within 3D-printable biomaterials, or design mechanisms that amplify local actuation with material-scale FEA. Furthermore, other unit cell designs (e.g., hybrid or a new nodal reentrant) can be incorporated following the same type of analysis.

Unit cells can be chosen to have different combinations of properties (E^{A}≠E^{N}, v^{A}≠−v^{N}) using our equations. More than two unit cells can be combined in new patterns, for example tetrachiral patterns, to add twisting motions. Three dimensional geometries, which we have investigated with fixed connections between each node, enable even further variation with different types of connections. The shared nodes of these lattices create a grid that enables novel custom metamaterials with deformations that can be mechanically “programmed” piece by piece.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, we claim:

1. A hybrid metamaterial structure comprising:
at least one auxetic portion; and
at least one non-auxetic portion connected to the auxetic portion, the at least one non-auxetic portion having a nodal honeycomb unit geometry.

2. The structure recited in claim 1, wherein the nodes are arranged in rows and columns.

3. The structure recited in claim 1, wherein the at least one auxetic portion has an anti-tetrachiral unit geometry.

4. The structure recited in claim 1, wherein the nodal honeycomb unit geometry comprises individual honeycomb cells connected by rectangular nodes.

5. The structure recited in claim 1, wherein the nodal honeycomb unit geometry comprises individual honeycomb cells connected by square nodes.

6. The structure recited in claim 1, wherein the nodal honeycomb unit geometry comprises individual re-entrant honeycomb cells connected by rectangular nodes.

7. The structure recited in claim 1, wherein the at least one auxetic portion and the at least one non-auxetic portion cooperate to form a planar member.

8. The structure recited in claim 1, wherein the at least one auxetic portion and the at least one non-auxetic portion cooperate to form a tubular member.

9. A stent comprising the structure recited in claim 1.

10. A soft robot component for tunnel traversing comprising the structure recited in claim 1.

11. A gripping component for manufacturing comprising the structure recited in claim 1.

12. The structure recited in claim 1, wherein the auxetic and non-auxetic portions are asymmetrically arranged about the structure.

13. The structure recited in claim 1, wherein the auxetic and non-auxetic portions have equal Young's Modulus values and equal but opposite Poisson's Ratio values.

14. The structure recited in claim 1, wherein the auxetic and non-auxetic portions are arranged in a checkerboard pattern to form a flexural member.

15. The structure recited in claim 1, wherein the auxetic and non-auxetic portions are arranged in an alternating pattern along the length of the structure to form an expansional member.

16. The structure recited in claim 1, wherein the nodes interconnecting the unit cells of the non-auxetic portion are equally spaced along the same axis.

17. The structure recited in claim 1 comprising a planar sheet rolled into a tube and interconnected along longitudinal edges at the nodes.

18. The structure recited in claim 1, wherein the auxetic and non-auxetic portions cooperate to form layers connected to one another in a stack.

19. The structure recited in claim 18, wherein the layers comprise a first layer formed only from auxetic portions and a second layer formed only from non-auxetic portions.

20. The structure recited in claim 18, wherein the planar members comprise first and second layers formed only from auxetic portions and third and fourth layers formed only from non-auxetic portions, the third and fourth layers being directly connected together and positioned between the first and second layers.

21. The structure recited in claim 18, wherein the layers are planar.

22. The structure recited in claim 18, wherein the layers are non-planar.

23. The structure recited in claim 18, wherein the layers are tubular and stacked in a concentric manner.

24. The structure recited in claim 18, wherein pins connect aligned nodes of adjacent layers.

25. A hybrid metamaterial structure comprising:
at least one auxetic portion having an anti-tetrachiral unit geometry; and
at least one non-auxetic portion connected to the auxetic portion and having a re-entrant honeycomb unit geometry interconnected by square nodes, wherein the auxetic and non-auxetic portions have equal Young's Modulus values and equal but opposite Poisson's Ratio values.
